(19) United States
(12) Patent Application Publication
(10) Pub. No.: US 2004/0028759 A1
(43) Pub. Date: Feb. 12, 2004

(54) Pharmaceutical or dietary composition containing a vegetable oil, in particular olive oil and sitosterol

(76) Inventors: Jean-Claude Maurel, Castries (FR); Gerard Cros, Montpellier (DE); Gerard Ribes, Montpellier (FR)

Correspondence Address: DENNISON, SCHULTZ & DOUGHERTY, 1745 JEFFERSON DAVIS HIGHWAY, ARLINGTON, VA, 22202, US

(21) Appl. No.: 10/415,092

(22) PCT Filed: Oct. 26, 2001
(86) PCT No.: PCT/EP01/12757

(30) Foreign Application Priority Data
Oct. 31, 2000 (GB) 0026609.8

(51) Int. Cl. A61K 35/78

(52) U.S. Cl. 424/769

(57) ABSTRACT

The present invention relates to a pharmaceutical or dietary composition containing, as active principle, a mixture comprising:

a vegetable oil naturally containing more than 10 μg, preferably more than 50 μg, of vanadium per litre of oil and in which the proportion of oleic acid with respect to the whole of the fatty acids is at least 20% by weight and preferably at least 30% by weight and,

sitosterol,

said composition containing from 0.5 to 100 parts by weight of vegetable oil per part by weight of sitosterol.

In said composition, said vegetable oil is preferably olive oil.

The compositions of the present invention are particularly intended for treating insulin resistance and its complications, as well as hypercholesterolaemia and hypertriglyceridaemia.

The present invention relates to novel pharmaceutical or dietary compositions which contain a vegetable oil, in particular olive oil, and sitosterol.

International application WO 96/23811 describes organometallic complexes which are obtainable by a reaction of three types of compound, namely a metal cation, in particular a vanadium cation, β- or γ-sitosterol, and a mono-, a di- or a triglyceride of the formula below:

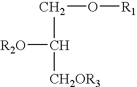

in which:

R₁ is an acyl residue of a saturated or unsaturated C₁₄-C₂₄ fatty acid, hydrogen, or a mono-, di-, or trigalactose or glucose;

R₂ is an acyl residue of a C₁₈ fatty acid having one unsaturated bond, preferably an oleic acid residue, or one of its positional isomers with respect to the double bond (cis-6,7,9,11 and 13) or one of its iso-branched isomers; and

R₃ is an acyl residue of a saturated or unsaturated C₁₄ to C₂₄ fatty acid, or a hydrogen atom.

The complexes described in the document WO 96/23811, and more particularly the complexes in which the metal cation is vanadium, have proved to be particularly interesting for the treatment and/or prevention of insulin dependent or non-insulin dependent diabetes, of the cardiovascular complications thereof and/or of insulin resistance and of the cardiovascular complications thereof, in particular arterial hypertension, obstructive coronary pathologies (myocardial infarction, and/or, of ocular or peripheral microangiopathies), and/or hypercholesterolaemia and/or hypertriglyceridaemia, as well as android-type obesity.

It has also been demonstrated, in the International application, that, insofar as an oil is used which is sufficiently rich in oleic acid, it was possible to prepare a dietary product by introducing, into this oil, sitosterol, in particular in the form of a plant extract containing it, and a vanadium salt in which the vanadium is in an oxidation state of 4 or 5, in particular a vanadate or vanadium acetylacetonate.

The International application WO 98/01461 describes complexes obtained by a reaction of three types of compound, namely a metal cation, in particular vanadium, sitosterol, sitostanol or a mixture of sitosterol and sitostanol and a diglyceride of the formula below:

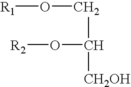

in which:

R₁ is an acyl residue of oleic acid (C_{18:1}),

R₂ is an acyl residue of a saturated or unsaturated, linear or branched fatty acid having between 2 and 18 carbon atoms.

It appears, in the text of this International application that the complexes which are described therein have an increased activity with respect to those described in the application WO 96/23811.

Furthermore, insofar as the diglycerides, the use of which is described for the preparation of these complexes are present in various vegetable oils, in particular in olive oil, it emerges from this application that it is possible to prepare dietary compositions from olive oil, sitosterol and/or sitostanol and a vanadium compound.

The Applicant has now noticed that, on the condition of starting with an oil which is naturally sufficiently rich in vanadium, it is possible, by introducing sitosterol into this oil, to prepare pharmaceutical or dietary compositions which, at an equal concentration of metallic vanadium, have activities which are clearly greater than those described above in which the vanadium was in general added in the form of a salt.

Thus, the present invention relates to a pharmaceutical or dietary composition containing, as principle active, a mixture comprising:

of a vegetable oil naturally containing more than 10 μg, and preferably more than 50 μg, of vanadium per litre of oil and in which the proportion of oleic acid with respect to the whole of the fatty acids is at least 20% by weight and preferably at least 30% by weight and,

sitosterol,

said composition containing from 0.5 to 100 parts by weight of vegetable oil per part by weight of sitosterol.

The invention relates to both pharmaceutical compositions, i.e. compositions intended for use as medicaments, and to dietary compositions, in particular dietary products which can be used a food supplements.

These different types of product of the invention have, in common the fact of containing sitosterol and a vegetable oil which is rich in both oleic acid and vanadium, as active principles.

It is known that vegetable oils contain very variable contents of oleic acid Ca_{18:1} and this, in particular, as a function of the nature of the plant and of its geographical origin.

The vegetable oils used within the context of the present invention contain at least 20% by weight of oleic acid with respect to the whole of the fatty acids that they contain and, preferably, at least 30% by weight of oleic acid.

Furthermore, the oils used for the preparation of the compositions of the invention naturally contain more than 10 μg of vanadium per litre of oil, and, preferably, more than 50 μg of vanadium per litre of oil.

Thus, for the preparation of the compositions of the invention, it will be possible to select, in particular, palm oil or sunflower oil of oleic variety, which generally contain from 20 to 50% by weight of oleic acid, on the condition that these oils further contain, naturally, vanadium in a sufficient amount.

Olive oil will however be preferably selected as vegetable oil, which generally contains from 56 to 82% of oleic acid and certain varieties of which are particularly rich in vanadium.

Thus, the Applicant has noticed that while large-scale distribution oils generally contain less than 10 μg/l of vanadium, certain olive oils, in particular from the south of France, contain about 150 μg/l, while oils from Andalusia often contain 250 to 350 μg/l of vanadium.

The oils containing more than 50 μg/l of vanadium are more particularly interesting for the preparation of the compositions of the invention.

Oils containing more than 100 μg/l of vanadium and, preferably, more than 200 μg/l of vanadium, will however be preferably selected.

As set forth above, oils which are rich in oleic acid will be selected. Extra virgin oils, first cold pressing extra virgin oils, will preferably be selected, and in particular, as set forth above, olive oil will preferably be selected.

However, it will also be possible to select oleic variety sunflower oil for example, on the condition however that it contains sufficient vanadium.

The sitosterol incorporated in the compositions of the invention can be β- or γ-sitosterol or can be introduced in the form of a plant extract containing at least one of these two forms of sitosterol.

It will in particular be possible to use various commercial products.

More particularly, commercial sitosterol will be used which is extracted from soya.

In such a product, the sitosterol generally represents from 50 to 70% by weight of the product and is generally found in a mixture with campesterol and sitostanol in respective proportions in the order of 15% each.

Commercial sitosterol can also be used which is extracted from a variety of pine called tall oil.

In general, it will be possible to use the sitosterol in a mixture with sitostanol, on the condition however that the sitosterol represents at least 50% by weight of the mixture.

For the preparation of the compositions of the invention, as set forth above, an olive oil will preferably be selected, preferably a first cold pressing olive oil and preferably, an olive oil containing more than 60% by weight of oleic acid will be selected.

Such an oil will advantageously contain more than 50 μg/l of vanadium and preferably, more than 100 μg/l of vanadium.

The compositions of the invention contain the two active ingredients, namely a vegetable oil having a high content of oleic acid and of vanadium, and sitosterol, in proportions which can vary within broad limits. More specifically, the compositions of the invention contain from 0.5 to 100 parts by weight of vegetable oil per part by weight of sitosterol.

The proportions of these two constituents will in particular be determined as a function of the type of compositions sought after and, in particular, as a function of the application sought after. It will be possible in particular for the proportions to differ greatly, according to whether it is a purely pharmaceutical composition or a dietary composition.

Thus, relatively low proportions of vegetable oil will be selected. in particular proportions between 0.5 and 3 parts by weight of vegetable oil with respect to sitosterol, for the preparation of solid forms and, in particular, for the preparation of compositions in the form of gelatine capsules or soft capsules.

Intermediate proportions will be selected, e.g. proportions of 3 to 10 parts by weight of vegetable oil per part by weight of sitosterol, for the preparation of certain forms of food supplements.

Thus, it will be possible for example to prepare a margarine which incorporates a composition of the invention containing from 3 to 10 parts by weight of vegetable oil per part by weight of sitosterol.

Use of higher proportions of vegetable oil with respect to sitosterol will be made for the preparation of products in the form of a liquid. Such products will advantageously contain from 10 to 100 parts by weight of vegetable oil per part by weight of sitosterol.

The dietary or pharmaceutical compositions of the invention can further contain various pharmaceutical or food ingredients which are classically used.

Thus, for example, for solid forms, in particular for gelatine capsules or soft capsules, a hydrogenated vegetable fat, e.g. palm oil, which has the advantage of being liquid at 35° C., i.e. at the temperature of the pouring of the product into the gelatine capsules, and of being solid at ambient temperature, is added at will.

The solid forms of the compositions of the invention, whether they be for pharmaceutical or dietary use, can be in the form intended to disintegrate in the mouth, in particular in the form of a capsule which is chewed.

The solid forms of the compositions of the invention can also be in a form intended to be swallowed and to be disintegrated only in the intestine.

In this latter case, the composition will advantageously be contained in a soft capsule comprising a gastro-resistant coating which enables an intestinal absorption of the active principles.

The pharmaceutical compositions of the invention will advantageously be in the form of gelatine capsules or capsules.

However, it will be possible for the pharmaceutical composition of the invention to be in various forms and, in particular, in any form which is classically adapted to the encapsulation or the incorporation of a liquid composition in gelatine capsules.

The gelatine capsules or the capsules will preferably be covered with a gastro-resistant coating, so as to promote a better intestinal absorption.

As regards the dietary compositions, any form which is used classically in the field can be envisaged.

It will be possible for the dietary compositions of the present invention to be, in particular, in the form of an oil which is enriched with sitosterol and they will then be marketed in flasks or bottles or in any compatible packaging.

It will also be possible for the dietary compositions of the invention to be constituted of a food to which the sitosterol-enriched oil according to the invention is mixed. Cheeses will be cited in particular as examples of such mixtures incorporating the mixture of oil and sitosterol of the invention.

Another form which can be envisaged for the dietary compositions of the invention is a form of the type of that which can be used for pharmaceutical compositions, in particular, a composition in the form of gelatine capsules or capsules. As in the case of the pharmaceutical compositions, the dietary compositions presented in this form will advantageously be incorporated in capsules or gelatine capsules which are covered with a gastro-resistant coating.

According to another of its aspects, the invention also relates to a method of preparing the compositions of the invention.

According to this method, sitosterol is placed in solution in the vegetable oil.

This step of dissolution is advantageously carried out by moderate heating of the mixture, in particular by heating at a temperature in the order of 40° C., under agitation of the mixture.

As set forth above, the compositions of the invention are particularly intended for treating insulin resistance and its complications, as well as hypercholesterolaemia and hypertriglyceridaemia. The compositions of the invention have in particular enabled the level of vanadium used in this type of treatment to be considerably lowered.

The compositions of the invention have in particular enabled, in a long-lasting manner, the metabolic parameters to be improved, the glycaemia level to be stabilised, the circulating insulin level to be lowered and to be stabilised after the stopping of the treatment.

Furthermore, it has been possible for the serum cholesterol to be greatly lowered throughout the duration of the treatment and it has been possible for the triglycerides levels to be lowered and to be stabilised after the stopping of the treatment.

The following Examples are given in a manner which is purely illustrative of the present invention.

### A. EXAMPLES OF COMPOSITIONS ACCORDING TO THE INVENTION

### Example I

### Solid Composition

One part by weight of sitosterol per part by weight of olive oil containing more than 200 μg/l of vanadium and more than 70% of oleic acid and one part of hydrogenated palm oil, are mixed.

The mixture is agitated for ½ hour at 40° C. and then poured at the same temperature into gelatine capsules which are then sealed, and then covered with a gastro-resistant coating.

### Example II

### Food Form

One part by weight of sitosterol, three parts by weight of an olive oil containing more than 200 μg/l of vanadium and containing more than 70% of oleic acid and 20 parts by weight of hydrogenated vegetable oil, are mixed.

The mixture is agitated for ½ hour and then, after the addition of optional colorants and flavours, the product is allowed to regain ambient temperature.

### Example III

### Product in the Form of a Liquid

One part by weight of sitosterol is mixed with 50 parts by weight of an olive oil containing more than 200 μg/l of vanadium and containing more than 70% of oleic acid, the mixture is agitated for ½ hour at 40° C. and then bottled.

### B. PHARMACOLOGICAL TESTS

### B1. Demonstration of the Evolution of the Glycaemia in the Animal

a. Test Protocol

Male rats of the Wistar strain which originate from the rearing of the company Iffa-Credo and which weigh on average 160 g are kept for 4 days under observation and receive ad libitum food and drinking water. They are subjected to a temperature of 21° C.±1° C., and to a day/night cycle of 12 h.

They are then anaesthetised with ethyl ether and a dose of 60 mg/kg of Streptozotocine in solution in a pH 4.5 citrate buffer is administered to them by an injection made into the vein of the penis.

Three days later, the animals (which now weigh about 200 g) which have a glycaemia of between 3 and 4.9 g/l are grouped into batches of 6 animals, 3 per cage and are subjected to the treatment with the substance to be tested via intra-peritoneal injection (hereinafter referred to as IP) in solution in a fraction which is extracted from olive oil and which contains only triglycerides which behave in a neutral manner with respect to the products to be tested.

A batch of diabetic control rats is also made up, which, instead of the substances to be evaluated, receive the same volume of olive oil, via the IP route.

The measurement of the glycaemia is made at the desired time with a Glucometter III AMES (Bayer) on glucofilm, when it is a test of the evaluation of a hypogycaemic effect at 2 and 6 hours after the administration, or with a Glucometter I AMES, on glucostix when it is a treatment over several days, enabling evaluating the glycaemia regulatory role, preventive of the complications of diabetes and an eventual remanent effect after the stopping of the treatment, by incision of the tip of the tail and taking of a drop of blood.

b. Products Tested

The products tested in this Example all contain an olive oil containing more than 50 μg/l of vanadium and more than 60% by weight of oleic acid.

A control batch of rats receives injections of pure olive oil.

Four other batches of rats receive compositions in the form of a solution in olive oil. In this Example, three compositions according to the invention noted I, II and III are tested on three batches of six rats. These compositions contain, per one millilitre of olive oil:

composition I: 50 mg of sitosterol,

composition II: 65 mg of sitosterol,

composition III: 80 mg of sitosterol, respectively.

A composition which is identical to composition I but which further contains vanadium introduced at the rate of 1 μg (of metal) per millilitre of solution, is also tested in this Example.

This solution is noted IV.

The different solutions I to IV are tested in comparison with pure olive oil.

TABLE I

| | D0 | D4 | D8 | D11 |
|---|---|---|---|---|
| Olive oil control | 4.81 | 5.3 | 5.15 | 4.89 |
| Solution I | 4.77 | 3.8 | 3.5 | 3.19 |
| Solution II | 4.75 | 2.45 | 2.42 | 2.7 |
| Solution III | 4.81 | 2.5 | 2.22 | 2.28 |
| Solution IV | 4.82 | 2.83 | 2.68 | 2.58 |

### B2. Demonstration of the Activity of the Compositions of the Invention Upon Mixed Hyperlipidaemiae

a. Test Protocol

This test is carried out on the obese, non-diabetic Zucker rat having insulin-resistance, of genetic origin, under the following conditions:

Chronic treatment for 10 days (IP injection).

Study of the plasmatic lipids on 3 batches of animals:

8 rats receiving product A, the composition of which is the following:

100 ml of olive oil containing 200 μl of vanadium,

5 g of commercial sitosterol,

100 μg of vanadium in the form of vanadyl acetylacetonate VO(AcAc)₂

8 rats receiving product C, the composition of which is the following:

100 ml of olive oil containing 200 μg/l of vanadium,

5 g of commercial sitosterol,

8 <<reference>> rats receiving physiological serum (R), and restricted in food (consumption of drink and food is copied exactly, with a shift of 48 hours, from the consumption of batch A).

Individual cages.

Each animal receives, daily, between 9 and 10 a.m., an IP injection at the rate of 0.1 ml per 100 g weight.

The parameters studies are the following:

weight evolution,

food consumption,

water consumption/24 hours,

volume of urine/24 hours,

humoral content:

triglycerides

cholesterol (total, HDL, LDL, VLDL)

glycaemia

insulinaemia

b. Results

The obese Zucker rats are not diabetic at the beginning of their life, but they are insulin-resistant and have a pre-diabetes metabolism (polydipsia, polyphagia, polyuria).

From the study made, it emerges that the treatment by the product of the invention improves the metabolic parameters by a decrease in the consumption of water and food and above all by a very distinct and long-lasting decrease of the urinary volume.

The glycaemia is more stable in the treated animals.

The level of circulating insulin is lowered and stays stable after the stopping of the treatment.

The serum cholesterol is greatly lowered (30% on average) throughout the duration of the treatment and goes up a few days after the stopping of the treatment.

The triglycerides are very much lowered (30% on average), and remain at low values 20 days after the stopping of the treatment.

It should be noted that on this model, the statines lower the serum cholesterol by 20% on average and do not modify the triglycerides content.

Tables II to IV below illustrate the results obtained as regards the total cholesterol (Table II), the triglycerides (Table III) and the insulinaemia (Table IV), respectively.

In the Tables below, the values measured appear in bold type on a first line, and the standard deviations (SEM) figure on the following line.

TABLE II
Total cholesterol (in mmol/l)

| | | | | Treatment | | | | | | | |
|---|---|---|---|---|---|---|---|---|---|---|---|
| Chol | −8 | −6 | −4 | −1 | 2 | 6 | 8 | 10 | 15 | 23 | 29 |
| A | | 3.8 | 4.2 | 3.8 | 3.6 | 2.3 | 2.2 | 2.5 | 3.4 | 3.9 | 4.0 |
| | | 0.1 | 0.2 | 0.2 | 0.1 | 0.1 | 0.1 | 0.3 | 0.1 | 0.2 | 0.2 |
| C | | 3.8 | 3.9 | 4.0 | 3.8 | 2.4 | 2.2 | 2.0 | 3.1 | 3.9 | 4.5 |
| | | 0.2 | 0.1 | 0.1 | 0.2 | 0.2 | 0.1 | 0.2 | 0.1 | 0.2 | 0.2 |
| R | 3.7 | 3.7 | 3.9 | 3.5 | 3.3 | 3.0 | 3.2 | | 3.2 | 3.5 | 3.9 |
| | 0.2 | 0.1 | 0.2 | 0.2 | 0.3 | 0.2 | 0.2 | | 0.1 | 0.2 | 0.3 |

TABLE III
Triglycerides (in mmol/l)

| | | | | Treatment | | | | | | | |
|---|---|---|---|---|---|---|---|---|---|---|---|
| TG | −8 | −6 | −4 | −1 | 2 | 6 | 8 | 10 | 15 | 23 | 29 |
| A | | 3.5 | 2.8 | 2.5 | 0.8 | 2.0 | 2.2 | 2.3 | 1.8 | 2.8 | 2.8 |
| | | 0.3 | 0.2 | 0.4 | 0.1 | 0.2 | 0.2 | 0.1 | 0.1 | 0.2 | 0.2 |
| C | | 3.6 | 3.3 | 2.2 | 1.1 | 1.7 | 1.7 | 2.3 | 1.9 | 2.5 | 2.4 |
| | | 0.4 | 0.3 | 0.3 | 0.1 | 0.1 | 0.1 | 0.1 | 0.1 | 0.2 | 0.2 |
| R | 3.2 | 2.7 | 1.7 | 2.1 | 1.5 | 1.8 | 3.1 | | 2.5 | 3.2 | 2.8 |
| | 0.4 | 0.2 | 0.1 | 0.2 | 0.1 | 0.2 | 0.1 | | 0.2 | 0.3 | 0.3 |

TABLE IV
Insulinaemia (in mmol/l)

| | | | Treatment | | | | | | | |
|---|---|---|---|---|---|---|---|---|---|---|
| I | −5 | −3 | 0 | 3 | 7 | 9 | 11 | 16 | 23 | 30 |
| A | 9.7 | 12.8 | 12.9 | 19.6 | 10.4 | 9.1 | 6.1 | 10.8 | 9.9 | 12.2 |
| | 0.6 | 3.1 | 2.1 | 4.7 | 1.1 | 1.3 | 0.5 | 1.2 | 1.6 | 2.5 |
| C | 10.4 | 10.3 | 13.9 | 16.5 | 10.0 | 11.0 | 9.6 | 16.9 | 7.9 | 12.8 |
| | 1.0 | 0.4 | 1.5 | 3.0 | 1.2 | 1.6 | 2.0 | 3.3 | 0.6 | 2.8 |
| T | 12.0 | 10.7 | 7.5 | 8.4 | 5.9 | 5.3 | 6.6 | 23.8 | 11.4 | 7.6 |
| | 1.5 | 1.7 | 1.0 | 1.4 | 2.2 | 1.7 | 1.5 | 4.6 | 3.7 | 0.6 |

1. A pharmaceutical or dietary composition containing, as active principle, a mixture comprising:
a vegetable oil naturally containing more than 10 μg, preferably more than 50 μg, of vanadium per litre of oil and in which the proportion of oleic acid with respect to the whole of the fatty acids is at least 20% by weight and preferably at least 30% by weight and,
sitosterol,
said composition containing from 0.5 to 100 parts by weight of vegetable oil per part by weight of sitosterol.

2. The composition according to claim 1, characterised in that said vegetable oil contains more than 50 μg/l of vanadium.

3. The composition according to claim 1 or 2, characterised in that said vegetable oil contains more than 200 μg/l of vanadium

4. The composition according to one of claims 1 to 3, characterised in that said vegetable oil is a virgin oil, obtained by cold pressing.

5. The composition according to one of claims 1 to 4, characterised in that said vegetable oil is an olive oil.

6. The composition according to one of claims 1 to 5, characterised in that said oil is a first cold pressing olive oil.

7. The composition according to one of claims 1 to 6, characterised in that said vegetable oil is an olive oil containing more than 60% of oleic acid.

8. The composition according to one of claims 1 to 7, characterised in that it is in the form of a solid.

9. The composition according to claim 8, characterised in that it is in the form of gelatine capsules or soft capsules and contains from 0.5 to 3 parts by weight of vegetable oil as defined above with respect to sitosterol.

10. The composition according to claim 9, characterised in that said gelatine capsule or soft capsule is covered with a gastro-resistant coating.

11. The composition according to one of claims 1 to 10, characterised in that it contains from 3 to 10 parts of vegetable oil with respect to sitosterol.

12. The composition according to one of claims 8 to 11, characterised in that it further comprises a hydrogenated vegetable fat.

13. The composition according to one of claims 1 to 7, characterised in that it is in the form of a liquid and comprises from 10 to 100 parts by weight of vegetable oil per part by weight of sitosterol.

14. A method of preparing a composition according to one of claims 1 to 13, characterised in that it comprises a step of dissolving the sitosterol in a vegetable oil.

15. Use of a composition according to one of claims 1 to 13 for preparing a pharmaceutical or dietary composition intended for treating insulin resistance and the complications thereof or hypercholesterolaemia or hypertriglyceridaemia.
